(19) United States
(12) Patent Application Publication
(10) Pub. No.: US 2022/0287626 A1
(43) Pub. Date: Sep. 15, 2022

(54) A METHOD AND A SYSTEM FOR DETERMINING A LIKELIHOOD OF PRESENCE OF ARTHRITIS IN A HAND OF A PATIENT

(71) Applicant: HAVApp Pty Ltd, Nedlands, Western Australia (AU)

(72) Inventor: Mark Daly Reed, Nedlands, Western Australia (AU)

(21) Appl. No.: 17/636,255

(22) PCT Filed: Aug. 19, 2020
(86) PCT No.: PCT/AU2020/050861

§ 371 Date: Feb. 17, 2022

(30) Foreign Application Priority Data
Aug. 20, 2019 (AU) 2019903027

(51) Int. Cl. A61B 5/00; A61B 5/107; G06T 7/00; G16H 50/30; G06N 3/02

(52) U.S. Cl. CPC A61B 5/4528; A61B 5/004; A61B 5/1071; A61B 5/1072; G06T 7/0012; G16H 50/30; G06N 3/02; A61B 5/1079; A61B 2576/02; G06T 2207/20076

(57) ABSTRACT

A method of determining a likelihood of presence of at least one type of arthritis in a hand of a patient is provided, the method comprising capturing an image of a hand of the patient, processing the hand image to determine at least one first predictive value indicative of presence or absence of arthritis in the hand based on presence or absence of a plurality of identifiable hand features in the hand image, the identifiable hand features including visible physical hand features that are usable to diagnose arthritis in the hand, receiving patient information from the patient, the patient information comprising a plurality of responses to a plurality of respective questions that are relevant to diagnosing arthritis in the hand, and determining a likelihood of presence of at least one type of arthritis in the hand using the at least one first predictive value and the patient information.

### TECHNICAL FIELD

The present invention relates to a method of determining a likelihood of presence of at least one type of arthritis in a hand of a patient, and more particularly, although not exclusively, to a method of determining a likelihood of the patient having osteoarthritis and/or inflammatory arthritis in his/her hand.

### BACKGROUND

Different types of arthritis can affect the hands of an individual and cause joint pain, swelling, stiffness and deformity. The most common forms of hand arthritis are osteoarthritis and inflammatory arthritis, the latter group of which includes rheumatoid arthritis and psoriatic arthritis. Rheumatoid and psoriatic arthritis are forms of autoimmune disease, which can result in joint damage and disability. Early detection and treatment has been shown to result in better outcomes for patients, although many patients experience a significant delay in diagnosis of inflammatory arthritis in particular.

There is a need for a flexible, reliable tool that would determine the probability of a patient having a type of arthritis in his/her hand in a convenient manner.

### SUMMARY

In accordance with a first aspect of the present invention, there is provided a method of determining a likelihood of presence of at least one type of arthritis in a hand of a patient, the method comprising:
capturing an image of a hand of the patient;
processing the hand image to determine at least one first predictive value indicative of presence or absence of arthritis in the hand based on presence or absence of a plurality of identifiable hand features in the hand image, the identifiable hand features including visible physical hand features that are usable to diagnose arthritis in the hand;
receiving patient information from the patient, the patient information comprising a plurality of responses to a plurality of respective questions that are relevant to diagnosing arthritis in the hand; and
determining a likelihood of presence of at least one type of arthritis in the hand using the at least one first predictive value and the patient information.

In one embodiment, each first predictive value is indicative of a first probability of a respective type of arthritis in the hand and the method further comprises using the at least one first predictive value and the patient information to determine a second predictive value indicative of the likelihood of presence of at least one type of arthritis in the hand.

In one embodiment, capturing the image of the hand is carried out using a camera.

In one embodiment, the method further comprises processing the hand image to identify a plurality of hand shape features indicative of whether the hand in the captured image is a right hand or a left hand.

In this embodiment, the method further comprises using the plurality of hand shape features to determine a hand predictive value indicative of a probability that the hand in the captured image is one of a right hand and a left hand.

In one embodiment, the at least one type of arthritis comprises osteoarthritis.

In a further embodiment, the at least one type of arthritis comprises inflammatory arthritis. In this embodiment, the at least one type of arthritis includes at least one of rheumatoid arthritis and psoriatic arthritis.

The plurality of identifiable hand features may comprise at least wrist swelling, bony swelling of finger joints, and/or soft tissue swelling of finger joints, and may further comprise at least one of: skin rash, finger nail features, hand joint deformities, and rheumatoid nodules.

The patient information may be provided in the form of a questionnaire. The plurality of questions may comprise one or more questions relating to symptoms of the at least one type of arthritis.

The method may comprise using a first trained model to carry out the processing of the hand image so as to identify the plurality of hand shape features and determine the hand predictive value.

The method may further comprise using a respective second trained model to carry out the processing of the hand image so as to determine the presence or absence of the plurality of identifiable hand features and determine each first predictive value.

The method may comprise using a third trained model to determine the second predictive value based on the at least one first predictive value and the patient information.

In this embodiment, the method comprises:
one-hot encoding the patient information to generate respective one-hot encoded patient data; and
inputting the one-hot encoded patient data and the at least one first predictive value into the third trained model to determine the second predictive value.

In accordance with a second aspect of the present invention, there is provided a system for determining a likelihood of presence of at least one type of arthritis in a hand of a patient, the system comprising:
at least one interface for receiving a captured image of a hand of the patient and for receiving patient information from the patient, the patient information comprising a plurality of responses to a plurality of respective questions that are relevant to diagnosing arthritis in the hand;
a hand image processor arranged to analyse the hand image to determine at least one first predictive value indicative of presence or absence of arthritis in the hand based on presence or absence of a plurality of identifiable hand features in the hand image, the identifiable hand features including visible physical hand features that are usable to diagnose arthritis in the hand; and
a hand arthritis determiner arranged to process the patient information and the at least one first predictive value to determine a likelihood of presence of at least one type of arthritis in the hand.

In one embodiment, the captured image of the hand is a photographic image of the hand.

The hand image processor may further be arranged to analyse the hand image to determine a plurality of hand shape features indicative of whether the hand in the captured image is a right hand or a left hand.

The system may further comprise a hand shape determiner arranged to use the plurality of hand shape features to determine a hand predictive value indicative of a probability that the hand in the captured hand image is one of a right hand and a left hand.

In one embodiment, the system further comprises a data storage arranged to store the received captured hand image and the received patient information.

In this embodiment, the hand image processor may further be arranged to retrieve the hand image from the data storage and the hand arthritis determiner may further be arranged to retrieve the patient information from the data storage.

The hand arthritis determiner may comprise:
a first prediction module arranged to determine the at least one first predictive value, each first predictive value being indicative of a first probability of a respective type of arthritis in the hand; and
a second prediction determiner arranged to use the at least one first predictive value and the patient information to determine a second predictive value indicative of the likelihood of presence of the at least one type of arthritis in the hand.

In this embodiment, the second prediction determiner may comprise a one-hot encoding module arranged to receive the patient information and to use the patient information to generate one-hot encoded patient data. The second prediction determiner may be arranged to use the one-hot encoded patient data and the at least one first predictive value to determine the second predictive value.

### BRIEF DESCRIPTION OF THE DRAWINGS

Notwithstanding any other forms which may fall within the scope of the disclosure as set forth in the Summary, specific embodiments will now be described, by way of example only, with reference to the accompanying drawings in which:

FIG. 1 shows a flow chart of a method of determining a likelihood of presence of at least one type of arthritis in a hand of a patient in accordance with an embodiment of the present invention;

FIG. 2 shows a schematic dorsal view of the anatomy of a hand and wrist of the human body;

FIG. 3 shows an example of patient information provided in accordance with an embodiment of the present invention;

FIG. 4 shows a block diagram of a system for determining a likelihood of presence of at least one type of arthritis in a hand of a patient in accordance with an embodiment of the present invention; and

FIGS. 5 to 8 show respective hand images of various patients illustrating visually identifiable hand features that are usable to diagnose arthritis in the hand.

### DETAILED DESCRIPTION OF SPECIFIC EMBODIMENTS

During a clinical physical examination, a Rheumatologist may be able to observe and identify visible physical features of a hand of a patient associated with different types of arthritis in the hand. For example:
bony swelling, which corresponds to a presence of bony growths or nodes at finger joints, is a typical feature of osteoarthritis;
soft tissue swelling, which corresponds to a swelling of tendons or ligaments in joints of the hand, is a typical feature of inflammatory arthritis;
evidence of osteoarthritis can also usually be observed at the carpometacarpal (CMC) joint at the base of the thumb, as well as the distal interphalangeal (DIP) joints, proximal interphalangeal (PIP) joints and the interphalangeal joint (IP) for the thumb;
rheumatoid arthritis may be observed at the joints in the wrist, metacarpophalangeal (MCP) joints and proximal interphalangeal (PIP) joints;
swelling of the proximal interphalangeal (PIP) joints may be seen in patients having osteoarthritis (bony swelling) as well as in patients having inflammatory arthritis (soft tissue swelling);
other identifiable hand features may include deformities in the fingers, skin psoriasis, which in combination with other features such as soft tissue swelling, fusiform digit swelling (dactylitis) and/or psoriatic nail changes, may be associated with psoriatic arthritis; and
gouty lumps (tophi) may be associated with gout.

Visual observations of a Rheumatologist however may vary from one Rheumatologist to another depending on the experience of the Rheumatologist and other factors. Also, some features of soft tissue swelling can be difficult to detect visually.

Embodiments of the present invention broadly relate to a method and a system that provide a single non-invasive and cost-effective tool for determining a likelihood of presence of at least one type of arthritis in a hand of a patient in a reliable and convenient manner. It will be understood that embodiments of the present invention are particularly relevant to patients having pain in the hands, and more specifically in the hand joints.

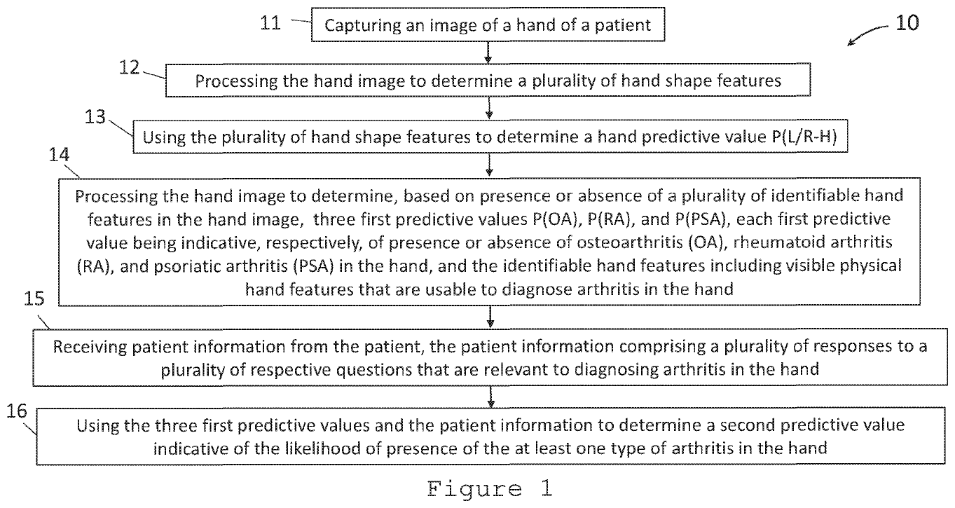

Referring to FIG. 1, there is shown a flow chart of a method 10 of determining a likelihood of presence of at least one type of arthritis in a hand of a patient. At step 11, the method 10 comprises capturing an image of a hand of the patient. The image of the hand is preferably a photographic image and may be captured using a capturing element, such as a camera. Further, for capturing the photographic image of the hand, the hand is preferably positioned on a substantially flat surface with a white background. For example, the hand may be positioned lying on a white table, or on a white sheet of paper on a support structure, such as a table. The hand may also be positioned lying on a white pillow for added comfort. It is also envisaged that an outline template may be provided for the hand to be positioned onto for capturing the hand image. At step 12, the method 10 comprises processing the hand image to determine a plurality of hand shape features indicative of whether the hand in the captured image is a right hand or a left hand. For example, the plurality of hand shape features may include an identification of fingers, a location of fingers, a size of each identified finger, an arrangement of fingers in space relative to each other, presence of a thumb and its position relative to the other fingers. It will further be understood that the plurality of hand shape features is not limited to such example and may include any other hand shape features which may be considered appropriate for providing an indication of whether the hand in the captured image is a right hand or a left hand.

The hand shape features are then used at step 13 to determine a hand predictive value P(L/R-H) indicative of a probability that the hand in the captured image is either a right hand or a left hand. For example, the hand predictive value P(L/R-H) may be compared to a hand threshold value wherein it is determined that the hand in the captured image is a right hand if P(L/R-H) is above the hand threshold value and a left hand if P(L/R-H) is below the hand threshold value, or vice versa. Additionally, the captured hand image may be processed to determine a plurality of shape features indicative of the presence of a hand and the plurality of determined shape features may be used to determine a presence predictive value indicative of a probability that the captured image comprises a hand. For example, the plurality of shape features may include presence of five fingers, presence of a wrist, and/or any other shape feature as considered appropriate by a person skilled in the art for the purpose of indicating the presence of a hand. The presence predictive value may be compared to a presence threshold value wherein it is determined that a hand is present in the captured image if the presence predictive value is above the given presence threshold value.

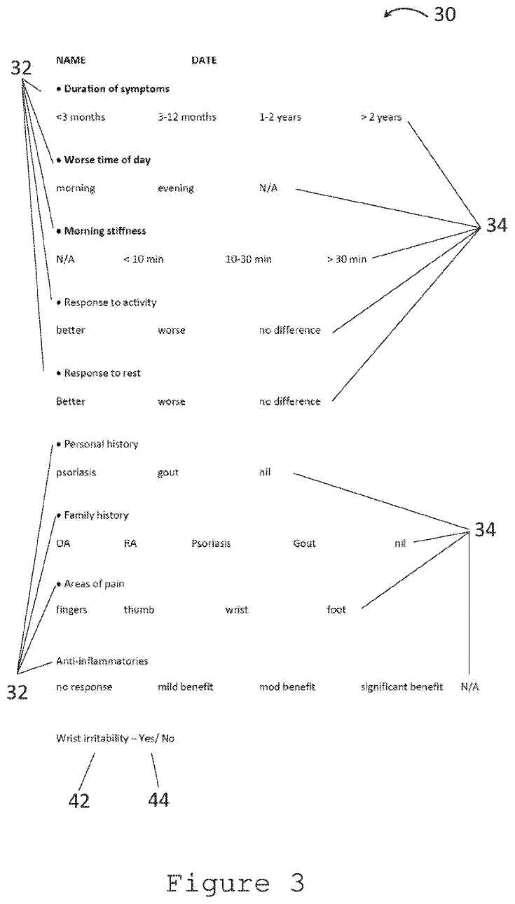

At step 14, the method further comprises processing the hand image to determine three first predictive values, P(OA), P(RA), and P(PSA), each predictive value being indicative of presence or absence of a respective type of arthritis in the hand of the patient. Specifically, the first predictive value P(OA) is indicative of a probability of osteoarthritis (OA) in the hand, P(RA) is indicative of a probability of rheumatoid arthritis (RA) in the hand, and P(PSA) is indicative of a probability of psoriatic arthritis (PSA) in the hand. The determination of the three first predictive values is based on presence or absence of a plurality of identifiable hand features in the hand photographic image, the identifiable hand features including visible physical hand features that are usable to diagnose arthritis in the hand. Steps 12 to 14 are generally automated and carried out using respective trained learning models, which will be described in more detail further below with reference to FIGS. 3 to 8.

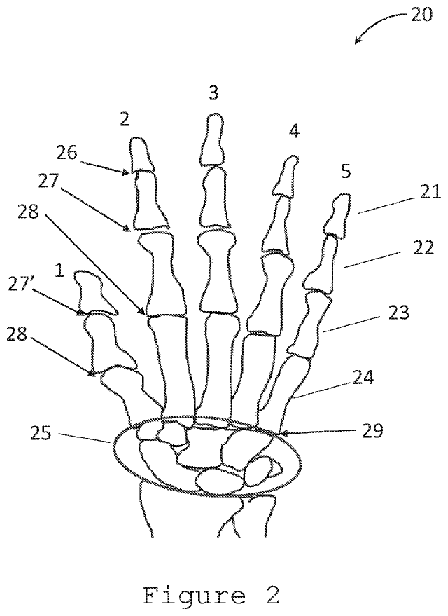

In the present described embodiment, the plurality of identifiable hand features comprises at least features of wrist swelling, bony swelling of the finger joints, and/or soft tissue swelling of the finger joints, which are known to Rheumatologists to be particularly useful to identify arthritis in a hand of a patient. To provide specific examples of identifiable hand features that are particularly useful to identify arthritis in a hand of a patient, it is convenient to refer to FIG. 2, which illustrates a schematic dorsal view 20 of the bone and joint anatomy of a hand and wrist of the human body. Finger numbers are indicated in FIG. 2 for further reference below. The bones that make up the hand, including the distal phalanges 21, the middle phalanges 22, the proximal phalanges 23, and the metacarpals 24 are shown in FIG. 2, as well as the bones that make up the wrist, i.e. the carpals 25. Importantly, the location of the joints that may typically be affected by arthritis are shown, specifically the DIP joints 26, the PIP joints 27 in fingers 2-5, the IP joint 27′ in thumb (finger 1), the MCP joints 28, and the CMC joints 29. For example, OA typically causes bony swelling, i.e. OA nodes, and bony swelling of the CMC joint at the base of the thumb as well as bony swelling of the DIP joints are typically useful indicators of OA. RA can cause wrist swelling as well as soft tissue swelling of the MCP joints. Both OA and IA can cause swelling of the PIP joints, bony swelling in OA, and soft tissue swelling in IA.

Other identifiable hand features, which presence or absence may be determined from the hand photographic image comprise at least one of skin rash, finger nail features, hand joint deformities, and rheumatoid nodules. Skin rash and finger nail features such as onycholysis and pitting, may be indicative of the existence of PSA. Rheumatoid nodules are typically indicative of the existence of RA.

It will be understood that the identifiable hand features are however not limited to these features and may further include any other feature that is capable of being used for diagnosing arthritis in a hand of a patient, such as tophi (urate crystal deposits), which may be indicative of the existence of gout, and dactylitis (fusiform swelling of a digit), which may be indicative of the existence of PSA. It will also be understood that embodiments of the present invention are not limited to a processing of the hand image to determine three first predictive values, and that the hand image may alternatively be processed to determine (i) one first predictive value only such as one of P(OA), P(RA), P(PSA), or (ii) two first predictive values, or (iii) more than three first predictive values, wherein for example, P(OA), P(RA), P(PSA), and an additional first predictive value indicative of a first probability of gout in the hand (P(G)) may be determined.

At step 15, the method 10 comprises receiving patient information from the patient, the patient information comprising a plurality of responses to a plurality of respective questions that are relevant to diagnosing arthritis in the hand. In accordance with a specific embodiment of the present invention, the patient information is received in the form of a questionnaire. FIG. 3 shows an example of a questionnaire 30 comprising a plurality of questions 32 and respective choices of responses 34 for the patient to complete. The questions 32 typically relate to the personal and family history of the patient and include questions relating to symptoms of the different types of hand arthritis. In accordance with embodiments herein described, the different types of hand arthritis include OA, RA, and PSA. However, questions relating to symptoms of one or more other types of hand arthritis may be included, for example, questions relating to symptoms of gout. Some questions may provide the ability to discriminate more strongly between different types of hand arthritis while other questions may be less discriminative. For example, the question ‘duration of symptoms’ is particularly useful in providing an indication of the presence or absence of OA, an answer ‘>2 years’ being particularly indicative of the existence of OA. The question ‘wrist irritability’ is particularly useful in providing an indication of the presence or absence of IA with an answer ‘Yes’ being particularly indicative of the presence of IA. Wrist irritability may be tested by a physical examination technique which consists in flexing the wrist passively to 90 degrees. If the patient experiences pain, then a Rheumatologist may consider this as an indication of the presence of IA rather than OA.

At step 16, the method 10 comprises using the three first predictive values and the received patient information to determine a second predictive value indicative of the likelihood of presence of the at least one type of arthritis in the hand of the patient. Step 16, like steps 12 to 14, is also generally automated and carried out using a trained learning model, which will be described in more detail further below with reference to FIGS. 3 to 8.

It will be understood that the first predictive values and the patient information may not be indicative of the presence or absence of the same type of arthritis in the hand of the patient. For example, the determined first predictive values may be indicative of presence of OA while the patient information may be indicative of presence of a sub-type of IA. Further, in some instances, such as a particular example of isolated inflammatory arthritis, the hand may not present any visible hand features indicative of the presence of a type of hand arthritis (i.e. IA in this example), in which setting the hand may be deemed to have a normal appearance and the determined first predictive values may be indicative of the non-existence of any type of hand arthritis. The patient information may however be indicative of presence of a type of hand arthritis, and in the particular example of isolated IA, be indicative of IA or more specifically a sub-type of IA. The method 10, by using a combination of the determined first predictive values and the patient information, can then provide a determination of the likelihood of presence of either OA or a sub-type of IA, or a combination of the conditions, in the patient.

In the present example, the method 10 can thus discriminate between three types of hand arthritis, i.e., osteoarthritis (OA) and two sub-types of inflammatory arthritis (IA) that are rheumatoid arthritis (RA) and psoriatic arthritis (PSA). As mentioned above, it will however be understood that embodiments of the present invention may further discriminate between less than three or more than three types of hand arthritis and may include types of hand arthritis that are different from OA, RA and PSA. For example, the method 10 may discriminate between any one or more of OA, RA, PSA and gout.

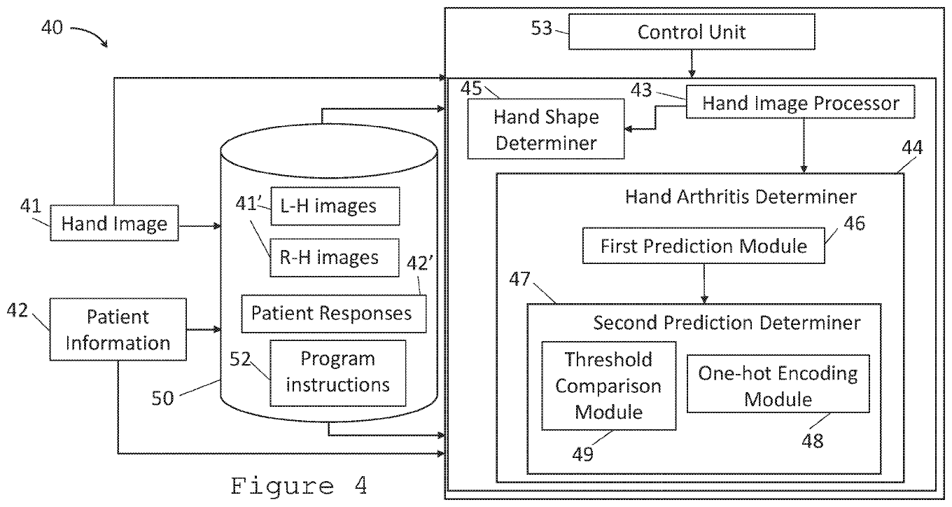

FIG. 4 is a detailed schematic block diagram of a system 40 provided in accordance with a specific embodiment of the present invention for implementing the method 10 of determining a likelihood of presence of at least one type of hand arthritis in a patient.

The system 40 comprises a first interface 41 for receiving the image of the hand of a patient captured at step 11 of method 10 illustrated in FIG. 1. The system 40 further comprises a second interface 42 for receiving the patient information from the patient at step 16 of the method 10. The captured image of the hand is a photographic image of the hand. The patient information comprises a plurality of responses to a plurality of respective questions that are relevant to diagnosing arthritis in the hand and is received in the form of a questionnaire as described above in relation to FIG. 3. It will be understood that a single interface may alternatively be used for receiving both the hand photographic image and the patient information. For example, an application, which may be web-based, for determining the likelihood of a type of hand arthritis in a patient may be installed on a mobile device such as a phone or a tablet equipped with a camera, and the patient (likely having pain in the hands, and more specifically in the hand joints) using the application may be prompted to capture an image of one of his or her hands and to provide responses to questions in a questionnaire such as questionnaire 30. In a specific embodiment, the patient may be prompted to capture an image of his or her right hand, or alternatively his or her left hand. The received hand image and patient information can then be processed ‘on the fly’ in real or near-real time. The system 40 further comprises a hand image processor 43 in communication with the interface 41 and arranged to carry out step 12 and part of step 14 of method 10. The hand image processor 43 is arranged to analyse the captured hand image to determine (i) a plurality of hand shape features indicative of whether the hand in the captured image is a right hand or a left hand, and (ii) information associated with presence or absence of a plurality of identifiable hand features indicative of the existence or non-existence of at least one type of hand arthritis. In the present specific embodiment, the system 40 further comprises a hand shape determiner 45 arranged to use the plurality of hand shape features to determine a hand predictive value P(L/R-H) indicative of a probability that the hand in the captured hand image is either a right hand or a left hand. Further, in the specific embodiment wherein the patient is prompted, when using the application, to capture an image of his or her right hand, the determined hand predictive value P(L/R-H) may be useful in confirming that the captured hand image is indeed an image of the patient's right hand. Similarly, in the specific alternative embodiment wherein the patient is prompted, when using the application, to capture an image of his or her left hand, and the determined hand predictive value P(L/R-H) may be useful in confirming that the captured hand image is indeed an image of the patient's left hand. In the example of the patient being prompted to capture an image of his or her right hand, if the hand predictive value P(L/R-H) is indicative that the hand in the captured hand image is indeed a right hand, then further steps 14-16 of the method 10 may be carried out. If the hand predictive value P(L/R-H) is however indicative of a probability that the hand in the captured hand image is a left hand, then the patient may not have correctly positioned his or her right hand relative to the camera and/or may have erroneously captured an image of the left hand, in which case the patient may choose or be prompted to capture another right-hand image. A similar process may be applied in the example of the patient being prompted to capture an image of his or her left hand wherein if the hand predictive value P(L/R-H) is indicative that the hand in the captured hand image is indeed a left hand, then further steps 14-16 of the method 10 may be carried out. If the hand predictive value P(L/R-H) is however indicative of a probability that the hand in the captured hand image is a right hand, then the patient may not have correctly positioned his or her left hand relative to the camera and/or may have erroneously captured an image of the right hand, in which case the patient may choose or be prompted to capture another left-hand image. In addition, as mentioned above, the captured hand image may be processed to determine a plurality of shape features indicative of the presence of a hand and the plurality of determined shape features may be used to determine a presence predictive value indicative of a probability that the captured image comprises a hand. If it is determined, based on a comparison of the presence predictive value to a presence threshold value that no hand is present in the captured image, the patient may then be prompted to capture another hand image.

The system 40 further comprises a hand arthritis determiner 44 that is in communication with the hand image processor and the interface 42 and is arranged to process the patient information and the information associated with the presence or absence of identifiable hand features to determine a likelihood of presence of at least one type of hand arthritis in the patient. The hand arthritis determiner 44 comprises a first prediction module 46 that is arranged to carry out step 14 of method 10 to determine the three first predictive values P(OA), P(RA), and P(PSA) based on the information associated with the presence or absence of identifiable hand features determined by the hand image processor 43. The hand arthritis determiner 44 further comprises a second prediction determiner 47 that is in communication with the first prediction module 46 and is arranged to implement step 16 of method 10, i.e. use the three first predictive values and the patient information to determine the second predictive value indicative of the likelihood of presence of at least one type of arthritis in the hand of the patient. The at least one type of arthritis in the hand in the present example includes OA, RA and PSA. The second prediction determiner 47 comprises a one-hot encoding module 48 in communication with interface 42 and that is arranged to receive the patient information from interface 42.

The one-hot encoding module 48 is then arranged to process the patient information to generate one-hot encoded patient data comprising only zeros and ones. The second prediction determiner 47 is then arranged to use the three first predictive values P(OA), P(RA), and P(PSA) and one-hot encoded patient information to determine the second predictive value. The sub-types RA and PSA are mutually exclusive and the second prediction determiner 47 may be arranged to compare P(RA) and P(PSA) to determine which of the first predictive values P(RA) and P(PSA) is the highest, i.e. the maximum value max(P(RA), P(PSA)). The value max(P(RA), P(PSA)) is used by the second prediction determiner 47 as a first predictive value P(IA) indicative of a probability of the patient having inflammatory arthritis, thereby combining both sub-types of IA. The second prediction determiner 47 may then be arranged to use the first predictive values P(OA) and max(P(RA, P(PSA)), and to process the first predictive values P(OA), max(P(RA, P(PSA)) and the one-hot encoded patient information to determine the second predictive value.

In one embodiment, the second prediction determiner 47 may additionally comprise a threshold comparison module 49 arranged to determine a respective threshold value between 0 and 1 for each first predictive value P(OA) and max(P(RA), P(PSA)) and to compare each of the first predictive values P(OA) and max(P(RA), P(PSA)) to the respective reference threshold value for determining whether each of the first predictive values P(OA) and max (P(RA), P(PSA)) is indicative of a positive or a negative diagnosis of the respective type of hand arthritis. The second prediction determiner 47 is then arranged to use the one-hot encoded patient information and the first predictive values weighed based on the comparison to the respective threshold values to determine the second predictive value. For example, if a respective reference threshold value for P(OA) is determined to be 0.5, and P(OA) has an actual value of 0.79, i.e. above the corresponding reference threshold value, then the threshold comparison module 49 considers that the first predictive value P(OA) is indicative that the patient is likely to have OA and the second prediction determiner 47 weighs P(OA) accordingly relative to max (P(RA), P(PSA)) and the one-hot encoded patient information to determine the second predictive value. Further, if a respective reference threshold value for max (P(RA), P(PSA)) is determined to be 0.5 and max(P(RA),P(PSA)) has a value of 0.23, i.e. below the corresponding reference threshold value, then the threshold comparison module 49 considers that the first predictive value max(P(RA),P(PSA)) is indicative that the patient is likely not to have IA and the second prediction determiner 47 weighs max (P(RA), P(PSA)) accordingly relative to P(OA) and the one-hot encoded patient information to determine the second predictive value.

Further, in another embodiment, it may also be envisaged that the threshold comparison module 49 be in communication with the one-hot encoding module 48 and that the one-hot encoding module 48 be further arranged to generate one-hot encoded first predictive data based on the comparison of the first predictive values P(OA) and max(P(RA), P(PSA)) to the respective determined reference threshold value. For example, if a respective reference threshold value is determined to be 0.5 and P(OA) has a value of 0.79, i.e. above the corresponding reference threshold value, then the one-hot encoding module 48 considers that P(OA) is indicative that the patient is likely to have OA and generates a one-hot encoded P(OA) having a value of 1. Further, if a respective reference threshold value is set at 0.5 and max(P(RA),P(PSA)) has a value of 0.23, i.e. below the corresponding reference threshold value, then the one-hot encoding module 48 considers that max(P(RA),P(PSA)) is indicative that the patient is likely not to have IA and generates a one-hot encoded P(IA) having a value of 0. The second prediction determiner 47 may then be arranged to use the one-hot encoded first predictive values P(OA) and P(IA) and the one-hot encoded patient information to determine the second predictive value.

It will also be understood that the patient information may be encoded in different forms other than one-hot encoded, and that an encoding module other than a one-hot encoding module may be used.

The system 40 further typically comprises a data storage 50 for storing a set of program instructions 52 executable by the hand image processor 43 and hand arthritis determiner 44. The data storage 50 further comprises one or more databases 41′, 42′ for storing, respectively, a plurality of hand images from one or more patients (including left-hand images and right-hand images) and a plurality of patient responses from one or more patients. For example, the captured hand image and patient information received by interfaces 41 and 42 may be stored in the respective databases 41′, 42′ after being processed for record purposes, for example, for other use by the Rheumatologist. Alternatively, it is also envisaged that the captured hand image and patient information received by interfaces 41 and 42 may not be processed ‘on the fly’ but may be stored in the respective databases 41′ and 42′ for processing at a later time. In this embodiment, the hand image processor 43 and hand arthritis determiner 44 may be arranged to retrieve, respectively, respective hand images and corresponding patient information from the data storage 50 at said later time and to process the retrieved respective hand images and patient information according to the steps of method 10 as described above.

It will also be understood that the data storage 50 may be a local data storage in the system 40 or may be a remote data storage on a server accessible over a communications network, such as the Internet.

The system 40 further comprises a control unit 53 in communication with the interfaces 41 and 42 and with the data storage 50 for controlling the operations of the hand image processor 43, the hand shape determiner 45 and the hand arthritis determiner 44.

In accordance with an embodiment of the invention, the hand image processor 43, the hand shape determiner 45, the first prediction module 46 and the second prediction determiner 47 are arranged to carry out steps 12 to 14 and 16 of method 10 using trained learning models. For example, the hand image processor 43, hand shape determiner 45 and first prediction module 46 may use trained deep learning models to carry out steps 12 to 14 and the second prediction determiner 47 may use a trained machine learning model.

The respective learning models used in accordance with embodiments of the present invention were trained on a plurality of captured hand images associated with a plurality of patients, and on patient responses received from a number of corresponding patients among the plurality of patients.

A description of the training processes for the learning models used in accordance with embodiments of the present invention will now be described.

### Deep Learning Model Training

A plurality of different image recognition approaches, such as custom or off-the-shelf deep learning or Convolution Neural Network approaches may be used to train the deep learning models. For example, ‘Transfer Learning’ approaches may be used and the inventors have found that the ‘Transfer Learning’ approach built on a “TensorFlow Inception v3” model (further referred to as “TensorFlow” model) was in particular suitable for the method 10 and system 40 in accordance with embodiments of the present invention. It will however be understood that other ‘Transfer Learning’ approaches, such as a ‘Transfer Learning’ approach built on a “Microsoft's Custom Vision Service (CVS)” may also be used.

For training the respective deep learning models, a plurality of hand images was captured from respective patients using a capturing device such as a camera. Each hand photographic image was captured with the hand positioned either lying on a white cushion in a substantially flat position or lying on a white sheet of paper on a table. Then, for each patient, both a photographic image of the patient's right hand and a photographic image of the patient's left hand were captured. The deep learning models were then trained on all right-hand and left-hand images. It is also envisaged that left hand images be mirrored to be “seen” by the models as right-hand images, wherein the deep learning models are trained on right-hand images only. Alternatively, it is also envisaged that right hand images be mirrored to be “seen” by the models as left-hand images, wherein the deep learning models are trained on left-hand images only.

Hand label data and diagnosis data associated with the respective right-hand photographic images and left-hand photographic images were further collected by a Rheumatologist. The hand label data relate to a plurality of hand shape features and identifiable hand features observed by the Rheumatologist in the respective photographic images. The diagnosis data were provided by the Rheumatologist based on the identifiable hand features observed in the captured hand images. Table 1 below provides an example of hand label data and associated diagnoses collected by the Rheumatologist in relation to a selection of hand photographic images, both right-hand images and left-hand images captured for a selection of respective patients with the respective hands positioned in a substantially flat position on a white A4 sheet of paper. In this example, the hand label data include a hand image number, whether the hand is a left hand or a right hand, the skin colour, presence or non-presence of OA nodes, wrist swelling, MCP swelling, PIP swelling and deformities. Numbers accompanying the PIP, MCP, CMC, and DIP descriptions are indicative of the finger number as illustrated in FIG. 2. It is to be noted that additional data relating to other hand features may further be provided such as, for example, data relating to skin rash, finger nail features, RA nodules, tophi and dactylitis.

TABLE 1
Data relating to visual observations of a Rheumatologist on captured hand images of
various patients.

| | | | | | Wrist | MCP | | |
|---|---|---|---|---|---|---|---|---|
| Image | Hand | Skin | Diagnosis | OA nodes | swelling | swelling | PIP swelling | Deformities |
| 4974 | L | W | OA | IP1, PIP2, PIP3, | | | | |
| | | | | PIP4, PIP5, | | | | |
| | | | | DIP2, DIP3, | | | | |
| | | | | DIP4, DIP5 | | | | |
| 4975 | R | W | OA | IP1, PIP2, PIP3, | | | | |
| | | | | PIP4, PIP5, | | | | |
| | | | | DIP2, DIP3, | | | | |
| | | | | DIP4, DIP5 | | | | |
| 7760 | R | W | RA | | Y | MCP1, | | |
| | | | | | | MCP2, | | |
| | | | | | | MCP3, | | |
| | | | | | | MCP4, | | |
| | | | | | | MCP5 | | |
| 7761 | L | W | RA | | Y | | | |
| 8727 | R | W | OA, RA | CMC1, DIP2, | Y | | | |
| | | | | DIP3, DIP4, | | | | |
| | | | | DIP5 | | | | |
| 8728 | L | W | OA, RA | DIP2, DIP4 | Y | | | |
| 9805 | R | W | PSA | | | | PIP4 | |
| 9806 | L | W | PSA | | | | PIP2, PIP3 | |

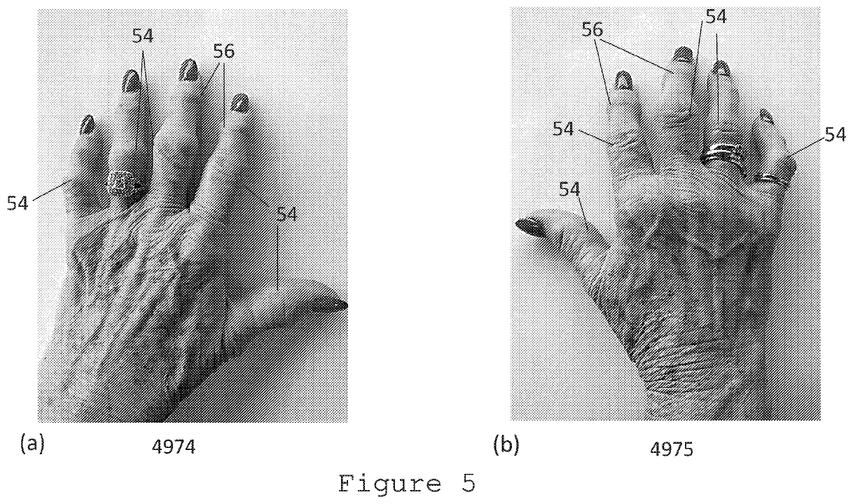

Captured hand images numbered 4974 and 4975 are respective left and right-hand images of a first patient, captured hand images numbered 7760 and 7761 are respective left and right-hand images of a second patient, captured hand images numbered 8727 and 8728 are respective left and right-hand images of a third patient, and captured hand images numbered 9805 and 9806 are respective left and right-hand images of a fourth patient. Images 4974 and 4975 listed in Table 1 are respectively illustrated in FIGS. 5(a) and 5(b), and for these images the Rheumatologist has provided a diagnosis of the patient having OA based on the respective visually identified hand features detailed in Table 1. For example, OA nodes 54 and 56 can clearly be observed in FIGS. 5(a) and 5(b), for example, at the finger joints IP1, PIP2, PIP3, PIP4, PIP5 and DIP2, DIP3.

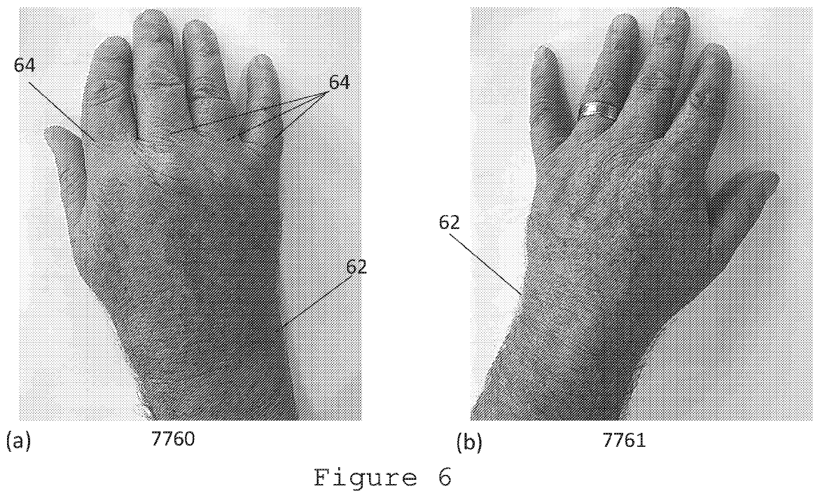

Hand photographic images numbered 7760 and 7761 are respectively illustrated in FIGS. 6(a) and 6(b), and for these photographic images the Rheumatologist has provided a diagnosis of RA based on the respective visually identified hand features detailed in Table 1. For example, wrist swelling 62 can be observed in FIGS. 6(a) and 6(b) as well as soft-tissue swelling 64 of the MCP joints of all fingers in the right hand of the patient (image 7760).

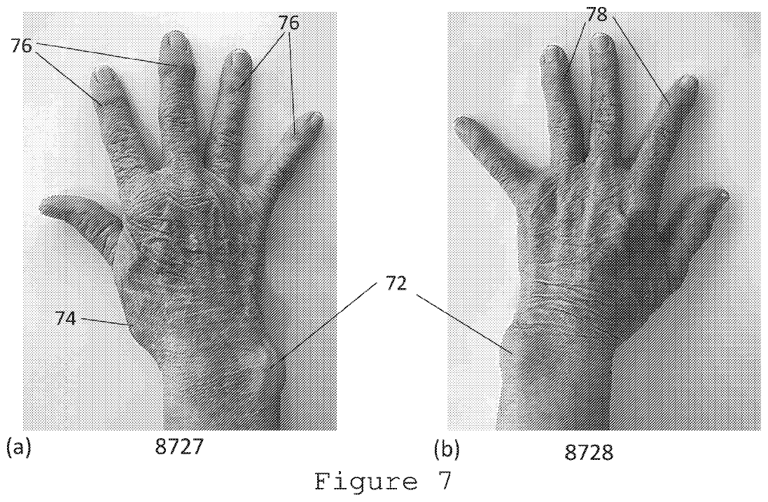

Hand photographic images numbered 8727 and 8728 are respectively illustrated in FIGS. 7(a) and 7(b), and for these photographic images the Rheumatologist has provided a dual diagnosis of the patient having OA and RA based on the respective visually identified hand features detailed in Table 1. For example, wrist swelling 72 in both the right and left hands can be observed in FIGS. 7(a) and 7(b), as well as an OA node 74 at the CMC joint of the right-hand thumb and OA nodes 76 at the CMC joints of fingers 2 to 5 in the right hand of the patient (FIG. 7(a)) and at the CMC joints of fingers 2 and 4 in the left hand of the patient (FIG. 7(b)).

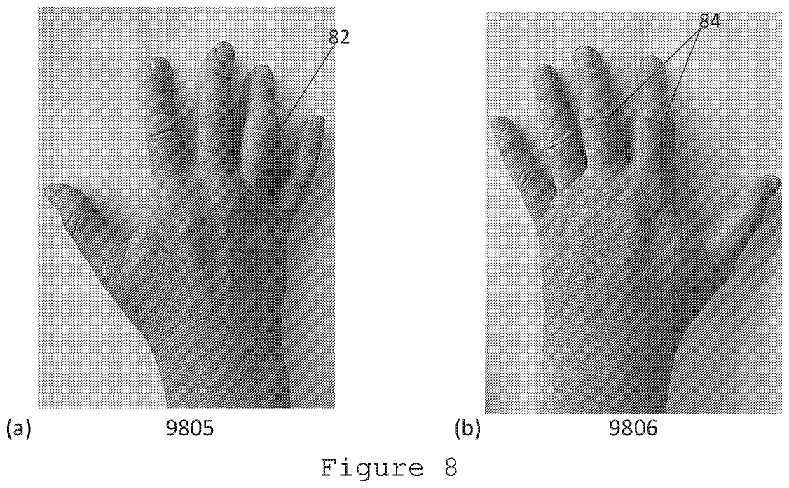

Hand photographic images numbered 9805 and 9806 are respectively illustrated in FIGS. 8(a) and 8(b), and for these photographic images the Rheumatologist has provided a diagnosis of PSA based on the respective visually identified hand features detailed in Table 1. For example, swelling of the PIP joint of finger 4 in the right hand of the patient can be observed in FIG. 8(a) while swelling of the PIP joints of fingers 2 and 3 in the left hand of the patient can be observed in FIG. 8(b).

Based on the combination of the captured hand images and associated hand label data and diagnosis, the training of the deep learning models then relies on image recognition and identification of patterns in the photographic hand images. The respective deep learning models develop a process whereby, using the captured left-hand and right-hand images input for deep learning model training purposes, the associated hand label data and the Rheumatologist's diagnosis, (i) features in newly captured hand images can be recognised, (ii) for each newly captured hand image, whether the hand is a right hand or a left hand can be determined, and (iii) a predictive value indicative of a type of hand arthritis can be output.

A very large dataset is preferable for the training of the models and in accordance with the present example, a total of 1010 hand photographic images were captured, and a corresponding number of associated hand label data and diagnosis data were collected. The 1010 hand images included both right-hand images and left-hand images of respective patients. Approximately 80% of these captured hand images and associated hand label data and diagnosis data were used for the training of the models and the remaining approximate 20% of captured hand images and associated hand label data and diagnosis data were withheld for further testing of the trained models.

Skin colour, as well as the presence of wrist jewellery such as watch and bracelet, rings on the fingers of the hand, and nail polish (not shown in Table 1) may be additional features capable of interfering with hand features recognition and pattern identification. A Machine Learning object detection approach may additionally be applied to the captured hand images to train object detection models for detecting such additional features and account for them during processing of the captured hand image. It is then envisaged that the trained object detection model be applied in a pre-processing step of the captured hand images prior to applying the deep learning models to the images, or that the trained object detection model be applied to the captured hand images in parallel to the deep learning models. Future iterations of this component may utilise object detection and/or image recognition to identify both the observable joint/skin/nail pathology, and additional features, such as jewellery.

In order to train the deep learning models, a notebook technology such as “Project Jupyter” was used to develop respective scripts, create, train and automate testing of the models. However, it will be understood that embodiments of the present invention are not limited to the training of deep learning models using “Project Jupyter” technology and that any other appropriate technology may be used to develop, create, train and automate testing of the models.

A separate model was developed for each determination of the hand predictive value and the respective first predictive values. Specifically, four different models were developed including (i) an L/R-H model that outputs a hand predictive value P(L/R-H) indicative of a probability that the hand in the captured hand image is either a right hand or a left hand, (ii) an OA model that outputs a first predictive value P(OA) indicative of a first probability that the hand in the captured image has OA, (iii) an RA model that outputs a first predictive value P(RA) indicative of a first probability that the hand in the captured image has RA, and (iv) a PSA model that outputs a first predictive value P(PSA) indicative of a first probability that the hand in the captured image has PSA. Each output hand predictive value and first predictive value corresponds to a value between 0 and 1.

Then, each output hand predictive value and first predictive value was compared to a respective reference threshold value between 0 and 1 to discriminate positive results and negative results. In a particular example, each reference threshold value was determined and set to a standard value of 0.5 and (i) an output hand predictive value greater than the corresponding reference threshold value was set to be indicative that the hand in the captured image is a right hand, (ii) an output hand predictive value smaller than the corresponding reference threshold value was set to be indicative that the hand in the captured image is a left hand, (iii) an output first predictive value greater than the reference threshold value was set to be indicative of a positive determination of the likelihood that the respective type of arthritis is present in the hand of the patient, and (iv) an output first predictive value lower than the reference threshold value was set to be indicative of a negative determination of the likelihood that the respective type of arthritis is present in the hand of the patient.

It will be however understood that the respective reference threshold values may have any another value different from 0.5 and may be determined and set to respective different values that may be equal, greater, or smaller than 0.5. Further, the respective reference threshold values may be adjusted to tune the determination of positive hand predictive value and first predictive values, as well as to tune the determination of true and false positives and true and false negatives. Also, it will be understood that the respective reference threshold value may alternatively be chosen such that an output hand predictive value greater than the corresponding reference threshold value is set to be indicative that the hand in the captured image is a left hand, and (ii) an output hand predictive value smaller than the corresponding reference threshold value is set to be indicative that the hand in the captured image is a right hand.

Each model was then tested using 160 hand images that were not used for the training process. RA and PSA are sub-types of inflammatory arthritis and are mutually exclusive. The separate RA and PSA models were accordingly tested simultaneously.

For testing purposes, the predictive power of each model was then evaluated using four test parameters:

1) Sensitivity, also known as recall, which measures the proportion of actual true positives that are correctly identified as such (e.g., the percentage of patients having a type of hand arthritis and who are correctly identified as having the condition based on the hand images):

$$Recall = \frac{{t}_{p}}{{t}_{p} + {f}_{n}}$$

wherein tₚ is the number of true positives and fₙ is the number of false negatives, i.e. the number of test results that wrongly indicate that a particular condition or attribute is absent.
2) Specificity, also known as true negative rate, i.e. the proportion of true negatives among all relevant negative results:

$$Specificity = \frac{{t}_{n}}{{t}_{n} + {f}_{p}}$$

wherein tₙ is the number of true negatives and fₚ is the number of false positives, i.e. the number of test results that wrongly indicate that a particular condition or attribute is present.
Precision, also known as positive predictive value, which measures the proportion of relevant “true” positive results among retrieved positive results:

$$Precision = \frac{{t}_{p}}{{t}_{p} + {f}_{p}}$$

3) Negative Predictive Value (NPV), which measures the proportion of test results that are “true” negatives among retrieved negative results:

$$N P V = \frac{{t}_{n}}{{t}_{n} + {f}_{n}}$$

The calculations of these four test parameters for each of the four models returned the results listed in Table 2 below.

TABLE 2
Test parameters output as a result of the
testing of each model created and trained
using the “TensorFlow” approach.

| Transfer | | | | |
|---|---|---|---|---|
| Learning | | | | |
| Approach | “TensorFlow” | | | |
| Models | L/R-H | OA | RA | PSA |
| Recall | 0.86 | 0.82 | 0.24 | 0.4 |
| Specificity | 0.66 | 0.52 | 0.79 | 0.88 |
| Precision | 0.68 | 0.81 | 0.34 | 0.26 |
| Negative | 0.85 | 0.55 | 0.69 | 0.93 |
| Predictive | | | | |
| Value | | | | |

As mentioned above, other deep learning or Convolution Neural Network approaches may be used. For example, ‘Transfer Learning’ approaches other than the “TensorFlow” approach may also be used to train the deep learning models and the inventors have also, for comparison, enquired the suitability of using the “Microsoft's CVS” approach to train the models. The predictive power of each model developed and trained using the “Microsoft's CVS” approach was also evaluated using the four test parameters mentioned above and calculations returned the following results listed in Table 3 below.

TABLE 3
Test parameters output as a result of the
testing of each model created and trained
using the “Microsoft's CVS” approach.

| Transfer | | | | |
|---|---|---|---|---|
| Learning | | | | |
| Approach | “Microsoft's CVS” | | | |
| Models | L/R-H | OA | RA | PSA |
| Recall | 0.53 | 0.71 | 0.12 | 0.07 |
| Specificity | 0.85 | 0.59 | 0.96 | 1.0 |
| Precision | 0.75 | 0.81 | 0.6 | 1.0 |
| Negative | 0.69 | 0.45 | 0.7 | 0.91 |
| Predictive | | | | |
| Value | | | | |

Based on the test results presented in Tables 2 and 3 above, the inventors concluded that the “TensorFlow” approach is more powerful in providing an indication of the existence or non-existence of a particular condition or attribute. Further, the “TensorFlow” model approach allows tuning model parameters such as the respective reference threshold values used for the determination of positives and negatives, as well as the Recall, Precision, Specificity, and NPV parameters. This provides the advantage that a final determination of the probability of the patient having one condition or attribute can be tuned depending on what the most important or relevant parameters are, for example Recall or Precision.

It will be understood that data provided in Tables 2 and 3 above are provided as an example only and may vary, for example, as the number of captured images increases and/or as the model parameters, such as the hand shape reference value and the respective reference threshold values, are adjusted.

### Machine Learning Model Training

In accordance with a specific embodiment of the present invention, the second prediction determiner 47 carries out the processing of the received patient information together with each first predictive value using a trained machine learning model.

The inventors have trialled a variety of different machine learning models including nearest-neighbours models, decision-trees, and Naïve-Bayes classifiers, and have found that three models produced results with a highest predictive power: (i) Support Vector Machines, (ii) Random Forests, and (iii) Logistic Regression.

Patient information was received from 280 patients associated with respective hand images out of the 1010 captured hand images. The patient information was received in the form of a plurality of responses to a plurality of respective questions in a questionnaire, such as questionnaire 30 shown in FIG. 3, the plurality of questions being relevant to diagnosing arthritis in the hand.

Most machine learning models cannot use data strings and require strictly numeric inputs. Thus, in order to train the machine learning model, the patient information was processed for generating data entries appropriately formatted for inputs to the machine learning model and further processing by the second prediction determiner 47.

Specifically, the patient information was initially processed to generate a survey dataset in the form of column entries. In a specific example shown in Table 4 below, a survey dataset is generated for a plurality of patients wherein, for each patient, the plurality of responses is provided in the form of a table with a column entry for each question. It is noted that Table 4 below provides an example only and does not reproduce all patient responses associated with all questions in the questionnaire 30. It will however be understood that the survey dataset will typically be generated to comprise as many rows as patients and as many columns as there are questions in the questionnaire.

TABLE 4
Survey dataset comprising patient responses to
respective questions provided in the form of column entries
for a plurality of patients.

| | | | Morning | Personal | Family | Wrist |
|---|---|---|---|---|---|---|
| Patient | Diagnosis | Image | stiffness | History | History | Irritability |
| XY | PSA, OA | 6372 | >30 min | N/A | None | Y |
| YY | OA | 6375 | N/A | N/A | OA | Y |
| XZ | OA | 6399 | N/A | | OA | N |
| YZ | OA, RA | 6404 | >10 min | | Psoriasis | Y |

Then, the survey dataset was one-hot encoded, using for example the one-hot encoding module 48 of system 40, so that the complete dataset used to train the machine learning model would only contain numeric inputs, and in the present specific example, only zeros and ones. The survey dataset was thus one-hot encoded wherein each column containing ‘n’ unique categorical inputs was converted into ‘n’ different columns filled with either 1 or 0. An exemplary representation of a one-hot encoding of the survey data in column ‘Wrist Irritability’ of Table 4 is shown in Table 5 below. After each variable in the survey dataset was appropriately one-hot encoded, the survey dataset contained 280 entries (i.e. rows) and 35 columns, each entry corresponding to a respective patient.

TABLE 5
One-hot encoding of the “Wrist Irritability”
column entries shown in Table 4.

| Wrist Irritability (Y) | Wrist Irritability (N) |
|---|---|
| 1 | 0 |
| 1 | 0 |
| 0 | 1 |
| 1 | 0 |

A machine learning model used in accordance with embodiments of the present invention to determine the likelihood of either OA or IA (i.e. RA or PSA) was then initially trained on the one-hot encoded survey dataset (280 entries in 35 columns) against a target diagnosis of either OA or IA.

The machine learning model trained on the survey dataset was then validated using Leave-One-Out-Cross-Validation (LOOCV), which is an ideal method of model validation in instances where the dataset used is relatively small and the corresponding training time for the model is similarly small. However, it will be understood that it is also envisaged that other methods of model validation be used as appropriate depending on the dataset provided.

The LOOCV method involves taking each entry in a dataset individually and excluding it (or leaving it out) while all the other entries in the dataset are used to train a given model. Once the model has been trained on the other entries, the model is then tested on the left-out entry.

In the present example, it means that, for the training and validation of the machine learning model, 279 entries out of the 280 one-hot entries were used while one entry was left out. Once the model was trained on these 279 entries, it was tested against the left-out entry. This process was then reiterated for each of the 280 entries in the dataset.

Further, the machine learning model was trained on a dataset incorporating the one-hot encoded survey dataset and the first predictive values P(OA), P(RA) and P(PSA) output using the trained “Tensor Flow” deep learning models. These first predictive values constitute numeric inputs for the second prediction determiner 47 and consequently do not necessarily need to be one-hot encoded. However, as mentioned, in one embodiment, the first predictive values may further be one-hot encoded based on a comparison to respective reference threshold values.

More specifically, the machine learning model was trained on a dataset incorporating the one-hot encoded survey dataset, the first predictive value P(OA) and the first predictive value P(IA), i.e. maximum value of P(RA) and P(PSA), which was used as a predictive value indicative of a probability that the patient has inflammatory arthritis, further taking in consideration the comparison of the first predictive values to the respective reference threshold values. Thus, two additional columns of first predictive values P(OA) and P(IA) were incorporated in a dataset along with the one-hot encoded survey dataset, wherein the two columns of one-hot encoded values P(RA) and P(PSA) were combined into a single column of values P(IA) according to the maximum value of P(RA) and P(PSA).

The model was thus subsequently trained on a dataset comprising a total of 280 entries and 37 columns and respective second predictive values were determined, for example using the second prediction determiner 47 of system 40, as respective outputs indicative of second probabilities of respective types of hand arthritis including one or more of OA, and IA. The relative weighing of the one-hot encoded survey dataset and output first predictive values in the trained machine learning model may be determined and adjusted based on the comparison of the first predictive values to the respective reference threshold values, which may further be based on how the first predictive values relate to the diagnosis provided by the Rheumatologist in the hand image dataset as illustrated in Table 1. Further, if the second predictive values were indicative of a likelihood of IA in the patient, the model was then trained to discriminate between RA and PSA based on the response to the questions ‘Personal History’ and/or ‘Family History’ in the questionnaire, wherein a positive response to ‘psoriasis’ would be indicative of the presence of PSA in the patient, while any other response to the questions ‘Personal History’ and/or ‘Family History’ would result in a likelihood of RA instead.

The machine learning model trained on the dataset including the one-hot encoded survey data and first predictive values was then also validated using the LOOCV as described above.

As mentioned above, several machine learning models were trialled by the inventors and trained as described above, and the three machine learning models (i) Support Vector Machines, (ii) Random Forests, and (iii) Logistic Regression were found to have the highest predictive power. The predictive power of each of the machine learning models was assessed by determining three test parameters, i.e. “accuracy”, “precision” and “recall” (also known as sensitivity as discussed above) and the results are shown in Table 6 below. Accuracy represents the proportion of model predictions that are actually, or “truly”, correct among the total number of model predictions.

TABLE 6
Results comprising the values of three test
parameters “Accuracy”, “Precision”, and “Recall” calculated
for each machine learning model trained and evaluated in
relation to each type of arthritis and based on the dataset
including the survey dataset and the first predictive values
output from the “Tensor Flow” model (i.e. based on the
captured image).

| | | Machine Learning Model used | | | | | |
|---|---|---|---|---|---|---|---|
| | | | | | | Support | |
| | | Logistic | | Random | | Vector | |
| | | Regression | | Forest | | Machine | |
| | | Type of Arthritis | | | | | |
| | | IA | OA | IA | OA | IA | OA |
| Accuracy | Survey + | 0.968 | 0.752 | 0.955 | 0.77 | 0.959 | 0.766 |
| | Image model | | | | | | |
| | outputs | | | | | | |
| Precision | Survey + | 0.972 | 0.753 | 0.956 | 0.771 | 0.956 | 0.844 |
| | Image model | | | | | | |
| | outputs | | | | | | |
| Recall | Survey + | 0.989 | 0.635 | 0.989 | 0.667 | 0.994 | 0.562 |
| | Image model | | | | | | |
| | outputs | | | | | | |

As can be seen in Table 6, the “Support Vector Machine” model trained on the combination of the survey dataset and “TensorFlow” model output first predictive values allowed predicting inflammatory arthritis with an accuracy, precision and recall of 95.9%, 95.6% and 99.4%, respectively, and it was additionally able to predict osteoarthritis with an accuracy, precision and recall of 76.6%, 84.4% and 56.2%, respectively.

Further, it is envisaged that the deep learning models and machine learning models used in accordance with embodiments of the present invention be trained on a crude 3D hand model created based on a plurality of images of a same patient's hand, the images being captured at respective different angles, or based on a recording of moving visual images of the patient's hand.

It is also envisaged that the trained models used to carry out processing of a newly captured hand image and associated patient information be further trained using the newly captured hand image and newly received associated patient information, whereby the accuracy, precision, and recall of the method and system in accordance with embodiments of the present invention can further be maximised.

Embodiments of the present invention present the advantage that anyone, and more specifically anyone having pain in the hands and/or in the hand joints, could have the possibility of self-obtaining a diagnosis for a type of hand arthritis ‘on the fly’. For example, using an application on a mobile device equipped with a camera, a person could simply capture an image of his or her hand using the camera and provide the link to this image in the application, or the person could alternatively be prompted by the application to capture an image of his or her hand. The person would further be prompted to provide responses to a series of questions in a questionnaire such as questionnaire 30. The response to the ‘Wrist Irritability’ question could be provided by the patient following a simple consultation at a General Practitioner, thereby avoiding the need for a referral to a Rheumatologist, which may be unnecessary in the first instance. It could also potentially be undertaken by the patients themselves, with a diagrammatic instruction, or under the supervision of an allied health care provider (Nurse, Physiotherapist, Occupational Therapist). One or more predictive values would then be output in the application and presented to the patient on the graphical interface of the mobile device, each of the one or more output predictive values being indicative of the likelihood of a respective type of hand arthritis. A more convenient and efficient screening of patients with hand arthritis would thus be allowed.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features in various embodiments of the invention.

Modifications and variations as would be apparent to a skilled addressee are determined to be within the scope of the present invention.

1. A method of determining a likelihood of presence of at least one type of arthritis in a hand of a patient, the method comprising:
capturing an image of a hand of the patient;
processing the hand image to determine at least one first predictive value indicative of presence or absence of arthritis in the hand based on presence or absence of a plurality of identifiable hand features in the hand image, the identifiable hand features including visible physical hand features that are usable to diagnose arthritis in the hand;
receiving patient information from the patient, the patient information comprising a plurality of responses to a plurality of respective questions that are relevant to diagnosing arthritis in the hand; and
determining a likelihood of presence of at least one type of arthritis in the hand using the at least one first predictive value and the patient information.

2. The method of claim 1, wherein each first predictive value is indicative of a first probability of a respective type of arthritis in the hand, and wherein the method further comprises using the at least one first predictive value and the patient information to determine a second predictive value indicative of the likelihood of presence of the at least one type of arthritis in the hand.

3. (canceled)

4. The method of claim 1, wherein the method further comprises processing the hand image to identify a plurality of hand shape features indicative of whether the hand in the captured image is a right hand or a left hand.

5. The method of claim 4, wherein the method comprises using the plurality of hand shape features to determine a hand predictive value indicative of a probability that the hand in the captured image is one of a right hand and a left hand.

6. The method claim 1, wherein the at least one type of arthritis comprises any one or both of osteoarthritis and inflammatory arthritis.

7. (canceled)

8. The method of claim 5, wherein the at least one type of arthritis comprises inflammatory arthritis and includes at least one of rheumatoid arthritis and psoriatic arthritis.

9. The method of claim 1, wherein the plurality of identifiable hand features comprises at least wrist swelling, bony swelling of finger joints, and/or soft tissue swelling of finger joints.

10. The method of claim 9, wherein the plurality of identifiable hand features further comprises at least one of: skin rash, finger nail features, hand joint deformities, and rheumatoid nodules.

11. The method of claim 1, wherein the patient information is provided in the form of a questionnaire and wherein the plurality of questions comprises one or more questions relating to symptoms of the at least one type of arthritis.

12. (canceled)

13. The method of claim 5, wherein the method comprises using a first trained model to carry out the processing of the hand image so as to identify the plurality of hand shape features and determine the hand predictive value.

14. The method of claim 2, wherein the method further comprises using a respective second trained model to carry out the processing of the hand image so as to determine the presence or absence of the plurality of identifiable hand features and determine each first predictive value.

15. The method of claim 2, wherein the method comprises using a third trained model to determine the second predictive value based on the at least one first predictive value and the patient information.

16. The method of claim 15, wherein the method comprises:
one-hot encoding the patient information to generate respective one-hot encoded patient data; and
inputting the one-hot encoded patient data and the at least one first predictive value into the third trained model to determine the second predictive value.

17. A system for determining a likelihood of presence of at least one type of arthritis in a hand of a patient, the system comprising:
at least one interface for receiving a captured image of a hand of the patient and for receiving patient information from the patient, the patient information comprising a plurality of responses to a plurality of respective questions that are relevant to diagnosing arthritis in the hand;
a hand image processor arranged to analyse the hand image to determine at least one first predictive value indicative of presence or absence of arthritis in the hand based on presence or absence of a plurality of identifiable hand features in the hand image, the identifiable hand features including visible physical hand features that are usable to diagnose arthritis in the hand; and
a hand arthritis determiner arranged to process the patient information and the at least one first predictive value to determine a likelihood of presence of at least one type of arthritis in the hand.

18. (canceled)

19. The system of claim 17, wherein the hand image processor is further arranged to analyse the hand image to determine a plurality of hand shape features indicative of whether the hand in the captured image is a right hand or a left hand.

20. The system of claim 19, wherein the system further comprises a hand shape determiner arranged to use the plurality of hand shape features to determine a hand predictive value indicative of a probability that the hand in the captured hand image is one of a right hand and a left hand.

21. The system of claim 17, wherein the system comprises a data storage arranged to store the received captured hand image and the received patient information and wherein the hand image processor is further arranged to retrieve the hand image from the data storage and the hand arthritis determiner is further arranged to retrieve the patient information from the data storage.

22. (canceled)

23. The system of claim 17, wherein the hand arthritis determiner comprises:
a first prediction module arranged to determine the at least one first predictive value, each first predictive value being indicative of a first probability of a respective type of arthritis in the hand; and
a second prediction determiner arranged to use the at least one first predictive value and the patient information to determine a second predictive value indicative of the likelihood of presence of the at least one type of arthritis in the hand.

24. The system of claim 23, wherein the second prediction determiner comprises a one-hot encoding module arranged to receive the patient information and to use the patient information to generate one-hot encoded patient data.

25. The system of claim 24, wherein the second prediction determiner is arranged to use the one-hot encoded patient data and the at least one first predictive value to determine the second predictive value.
